(19) United States
(12) Patent Application Publication
(10) Pub. No.: US 2023/0158194 A1
(43) Pub. Date: May 25, 2023

(54) Ozonising module and remote management system of a plurality of such modules

(71) Applicant: O3ZY SA, Stabio (CH)

(72) Inventor: James Mossi, Stabio (CH)

(21) Appl. No.: 17/919,697

(22) PCT Filed: Apr. 21, 2021
(86) PCT No.: PCT/IB2021/053290

§ 371 Date: Oct. 18, 2022

(30) Foreign Application Priority Data
Apr. 21, 2020 (IT) 102020000008470
Mar. 30, 2021 (IT) 102021000007910

(51) Int. Cl. A61L 9/015

(52) U.S. Cl. CPC A61L 9/015; A61L 2209/14; A61L 2209/111; A61L 2209/212

(57) ABSTRACT

An ozonising module (1, 100) is disclosed having a housing (1a, 1b, 107) wherein an ozone generator assembly (3, 101) is accommodated, furthermore comprising
at least an ozone sensor (4, 102) connected to a control unit (C) apt to control said ozone generator (3, 101) based on a time scheduling and a comparison between an ozone threshold concentration and a concentration signal from said ozone sensor (4, 102),
ozone reduction means consisting of a filtering assembly with catalytic action (103) or a reducing assembly employing UV sources (113) through which an airflow is caused to flow by means of respective controlled ventilation means (103a).

### DESCRIPTION

### Background of the Invention

The present invention concerns an ozone-generating module and a system wherein such module can be installed.

### Field of the Prior Art

As known, for the sanitising of rooms, a number of different agents can be employed. Among these, the use is known of ozone (O₃) which, due to its strongly oxidising properties, is effectively used to neutralise any kind of living pathogenic or annoying organism. A vast literature exists on the effectiveness of ozone for sanitising rooms, also with respect to viral charges (for example against SARS-COV-2).

However, gaseous ozone is not stable in the long run and hence cannot be conveniently produced and stored in bottles for subsequent use. Therefore, for room sanitation operations, ozone is typically produced locally, according to requirements, through hand-held ozonisers which are managed by qualified operators. Thus ozone delivery occurs occasionally, upon request, for sanitising specific professional rooms (sterile chambers, hospital rooms, veterinary surgeries, ...), when necessary. These devices always require the intervention of qualified operators who suitably segregate the room which is being sanitised from the people normally occupying it. Non-manned ozonising devices evidently represent a risk for people’s safety.

However, there are circumstances in which it would be desirable to be able to have ozone available for permanently sanitising in a controlled manner both work and home spaces, without having to request the intervention of specialised services each time. In particular, up until today there is no ozoniser available which can be used effectively in work or home spaces and suitably controlled so as to permanently provide the desired sanitising effects, with no risks for the human beings occupying those environments for various reasons.

The need is therefore felt to provide a solution which allows to have an ozoniser available which is simple to install and use, which allows to maintain a secure and effective control on the sanitising of a certain number of spaces, with no risks for human beings or pets.

US2012/230879, EP2010828 and US2009/311138 describe apparatuses for sanitising environments with ozone.

However, the Applicant has detected that there is room for improvement in existing apparatuses, both in terms of structure and of operation.

### SUMMARY OF THE INVENTION

The object reported above is achieved, according to the present invention, with an ozonising module and a system wherein such module can be inserted, which has the features defined in the attached independent claims. Other preferred features of such solution are defined in the dependent claims.

### BRIEF DESCRIPTION OF THE DRAWINGS

Further features and advantages of the solution according to the present invention will nevertheless be more evident from the following detailed description of a preferred embodiment of the same, provided purely as a non-limiting example and illustrated in the attached drawings, wherein:

FIG. 1 is a perspective view of an exemplifying module according to a first embodiment of the invention;

FIG. 2 is a similar view to that of FIG. 1 with a removed cover;

FIG. 3 is a perspective view of the operating components of the module of FIG. 1;

FIG. 4 is a perspective view of the sole ozone-generating components of the embodiment of FIG. 1;

FIG. 5 is a perspective view, partly see-through, of a module according to an alternative embodiment of the invention;

FIG. 6A is an exploded perspective view of the module of FIG. 5;

FIGS. 6B and 6C are views according to different perspectives of the module of FIG. 5 with the covering lasing removed;

FIG. 7 is an exploded perspective view of all the components of the module of FIG. 5;

FIG. 8 is a graphic representation of a sanitising cycle according to the invention; and

FIG. 9 is a perspective view with parts removed of a variant of the embodiment of FIG. 5.

### DETAILED DESCRIPTION OF THE PREFERRED EMBODIMENTS

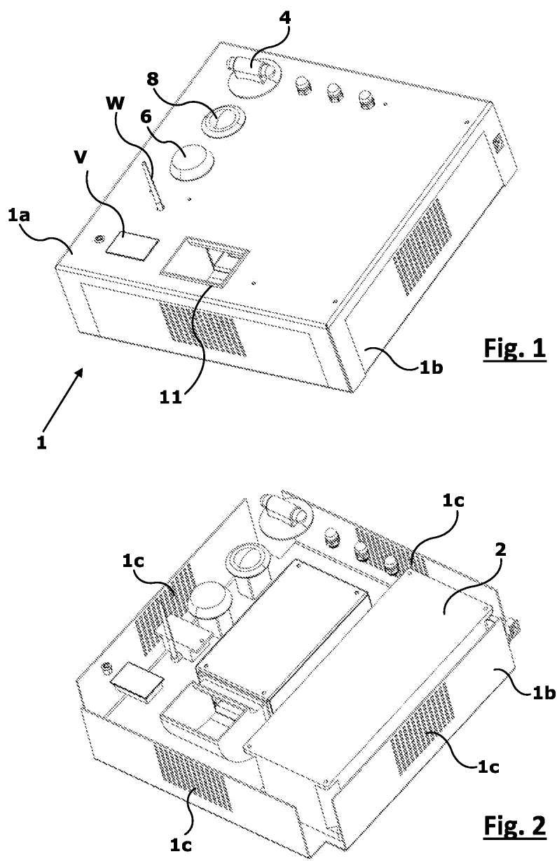

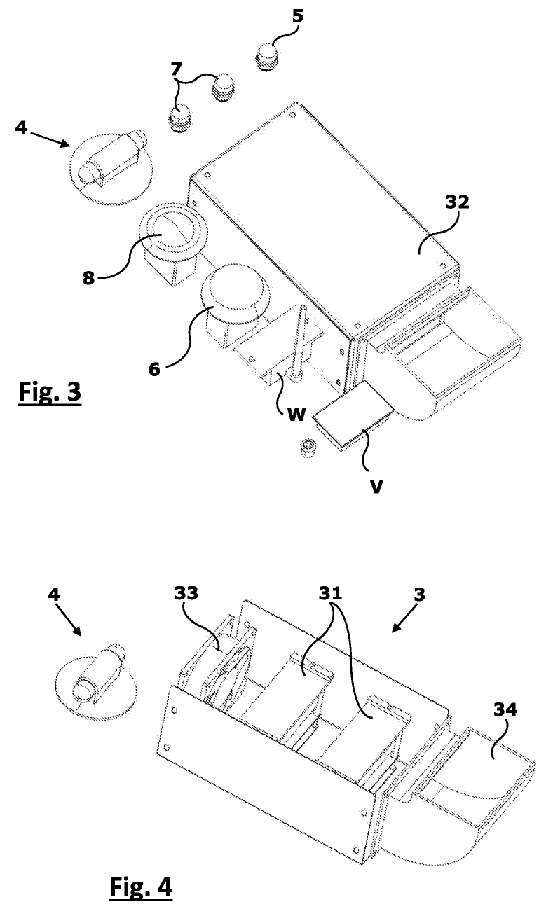

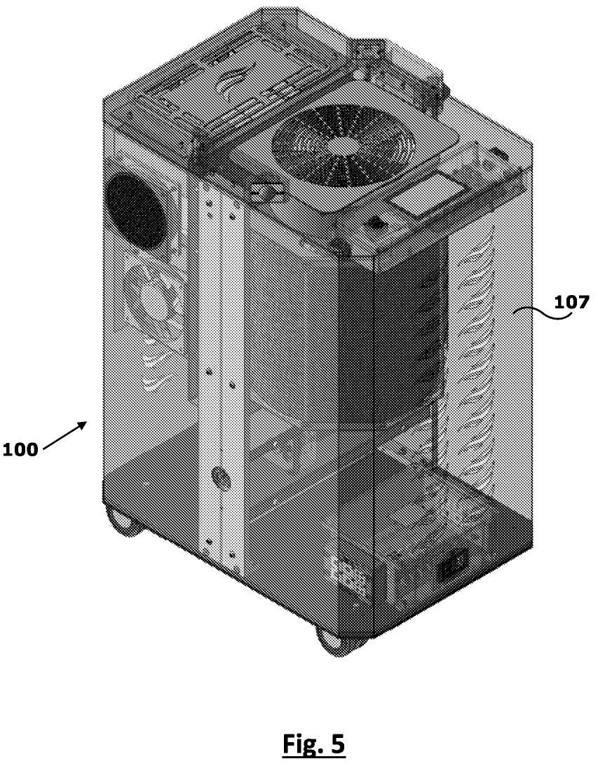

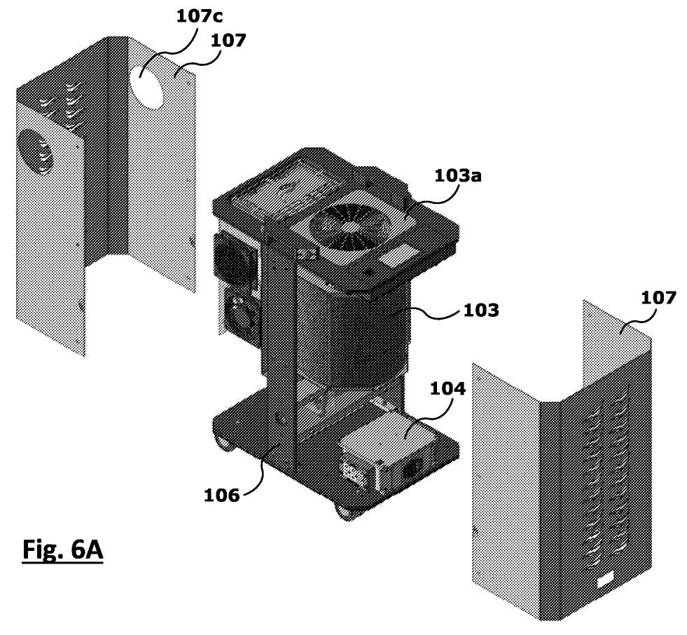

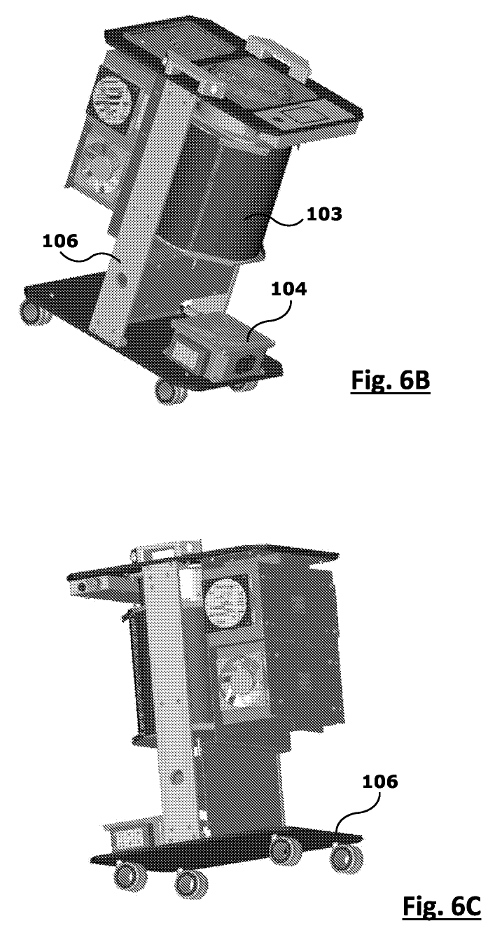

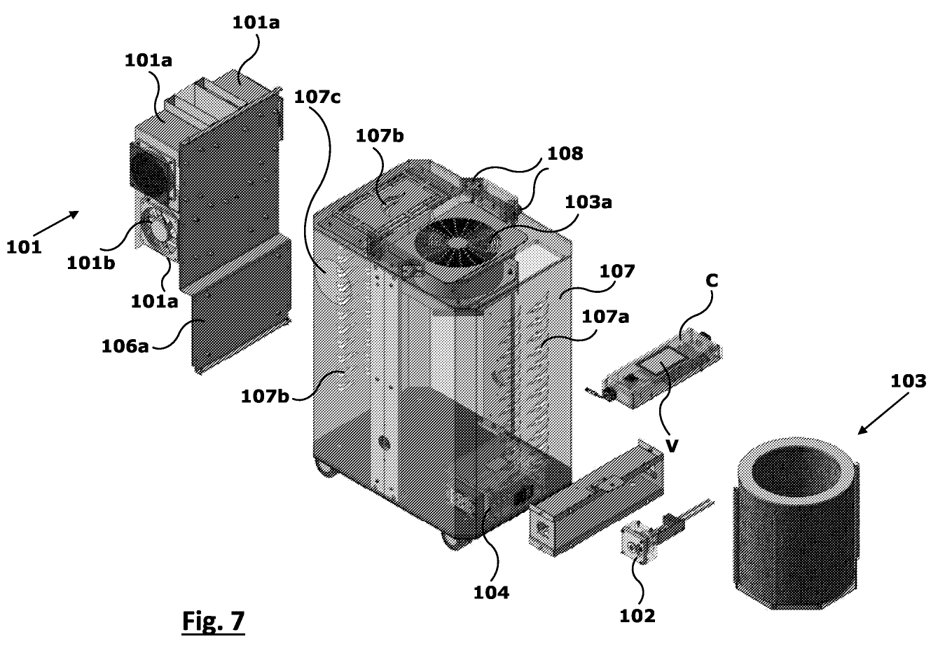

FIG. 1 shows an exemplifying ozonising module according to the invention. In a housing casing some operating components are housed, which partly run through a panel 1a of the casing, exposing to the outside a respective exposed part. As an alternative, all the operating components can be included in a common package or circuit board, mounted within module 1 as will be better described further on with reference to FIGS. 5-7: this layout can ease maintenance and repair operations.

Module 1 is of a shape and size apt to make the installation thereof simple in industrial or home spaces. For example, module 1 can be flush mounted in a standard frame of a modular suspended ceiling system, or it can provide a box mounting in the proximity of the floor, or a layout for wall-mounting or furthermore a flush mounting as an electrical household appliance in a piece of home furniture or, finally, suitably lined with finish covering (aesthetically pleasant) and support devices (feet or wheels) which make available as moving electrical household appliance, which can be manually moved across spaces, as illustrated in FIGS. 5-7.

In FIG. 2 panel 1a is removed, so that a closing carter 1b of case 1 remains visible, provided with lateral venting slits 1c, and the operating components of module 1.

Closing carter 1b and panel 1a can be made of bent metal sheet, but preferably of moulded or thermoformed plastic material, for example of ABS.

The operating components are powered by electric current, preferably through a power panel 2, also housed within the case 1 of the module, which transforms for example the network voltage (220 VAC in Europe) into a reduced voltage (for example 12 V) by which the various operating components work. Alternatively, it is possible to provide a stack of rechargeable batteries, for example of the LiFePo type, replaceable or kept in charge by a small charge maintenance power supply.

With reference to FIG. 4, the main operating components are a generator of gaseous ozone 3 and an ozone detection sensor 4.

Ozone generator 3 is preferably of the type with “corona plates”, which has proven particularly effective for this application in terms of the amount of ozone gas produced with the same electricity consumption and specific bulk of equipment; it is not ruled out to be able to employ another ozone production device.

In particular, generator 3 has a pair of corona plates 31, housed in a tubular box container 32 which defines a conveyor channel. At one end upstream of tubular container 32 a fan 33 is provided, suitably sized for cooling the system and push the ozone produced into the environment to be sanitised. In the example a fan with adjustable speed (preferably provided with a filter) is shown, of the type used for the cooling of computers but it is not ruled out to use also other types of mechanical fans, for example of a tangential type. Instead of a fan it is possible to provide other devices for the generation of an airflow, for example a controlled exit valve connected to a compressed air line. At the opposite end a deviator duct 34 (or equivalent flaps) is provided, which deviates an airflow to a cut-out 11 practised in panel 1a.

With this layout, an airflow is drawn from the environment through slits 1c of the module, pushed by fan 33 onto corona plates 31, thus enriched with ozone (O₃) and then pushed out again into the environment through cut-out 11.

Ozone sensor 4 is preferably analogical, for example of the type with Sn oxide with full scale of 10 ppm having an analogical output signal. However, it is not ruled out to be able to use sensors not only with other detection thresholds, but also with another detection principle and with other types of output signal, for example PWM or digital with serial communication.

According to the illustrated embodiment, ozone sensor 4 is mounted on panel 1a, in a spaced relationship from output cut-out 11. According to a preferred embodiment, there are provided at least two distinct sensors, to obtain detection redundancy and greater accuracy.

More preferably, at least a remote ozone sensor is provided, which is self-powered (for example by batteries, but also with an own power supply to be connected to a network socket) and installable in a remote position with respect to module 1, so as to detect the ozone concentration in environment positions remote from module 1. In this case, ozone sensor 4 is also provided with a wireless transmission unit (not shown), for example which transmits in WiFi with standard IEEE 802.11, for transferring the detected data to a receiver W onboard module 1, but other communication methods between the devices are not ruled out. In the drawings an antenna of receiver W is shown, designed to protrude from the panel 1a of module 1, but it is not ruled out that it may remain within module 1, hidden from view.

In addition to these main components, module 1 is provided also with a plurality of devices for the visual/sound signalling of the functional condition of the system. Preferably at least
a first light indicator 5 which makes evident the presence of electric voltage,
a second light and/or sound indicator 6 which indicates when ozone generator 3 is operating,
a third light indicator 7 which makes evident the condition or the ozone concentration level (excessive/tolerable);
a possible fourth light indicator for indicating operating anomalies.

These indicators can be alternatively all integrated on a single digital display V (for example a small LCD, touch-screen programmable display with alphanumeric and graphic functions) -possibly usable also for setting and activating the operation of the control unit of the apparatus - which changes viewing condition depending on which indicator it is necessary to show.

Optionally there are also provided:
a motion and/or presence detector 8, by which the presence in the room of people and/or animals above a certain size is detected,
a smoke presence sensor, usable for example for inhibiting the start of the module when smoke presence is detected, since ozone represents a combustion agent for the development of fires.

Like ozone sensor 4, also all the other operating components can be an integral part of the module or they can be mounted in a separate package and connected to module 1 in a wireless manner or through wires, or they can be both an integral part of module 1 or duplicates in a separate package and connected to the module to thus have a redundant control of the air condition and quality in the considered environment.

In addition to the main components mentioned above, module 1 comprises a control unit C with digital hardware (according to the illustrated embodiments it is located within power panel 2), preferably provided with programmable software or firmware, which collects the signals coming from the various sensors and suitably controls the operation of ozone generator 3. The control unit is preferably adjusted through a programming section (for example display V), by which at least the desired operation times and ozone level thresholds are set.

Since module 1 is provided with WiFi transmission capacity through receiver W or by wire (through Ethernet), it is possible to control and programme the control section also remotely, for example directly through a device with WiFi communicator (such as a tablet or a smartphone with appropriate application software) or remotely, acting through a hot-spot or a WiFi router connected to the Internet network and in communication with receiver W.

The overall operation of module 1 provides, after the start of the power supply, a preliminary security control step, wherein generator 3 is authorised to operate through a manual consent button and/or through the automatic consent deriving from lack of a presence signal from presence detector 8 (and possibly in the absence of smoke presence signals). Thereby, module 1 starts the production of ozone only when people and/or large animals (a cat or a dog, for example) are not expected to stay in the surrounding environment.

The device can be started also through time programming, managed by the user and designed to operate automatically, always taking into account the detectors and security sensors.

After which ozone generator 3 starts ozone production according to the way indicated above, until reaching a desired concentration, as determined by the signal coming from detection sensor 4. The ozone concentration level can be detected in real time or through a cyclic sampling.

The concentration threshold can be preset or set as desired by each individual user in the control section through the programming unit.

Preferably it is provided that ozonising module 1 operates according to a timed cycle (within which it is considered that no human being is present in the room) : thereby, through ozone sensor 4, it is possible to control ozone generator 3 so that it follows an ozone concentration profile with at least a rising ramp until the threshold level, with a plateau at the threshold concentration level, maintained for a time suitable to the sanitising but not to the stay of people and/or animals. At the end of this cycle, ozone sensor 4 remains constantly active and signals the change of the ozone concentration level. In such case, it is suitable for the module to be provided with presence sensor 8 or an equivalent thereof, to be able to immediately interrupt ozone production and emit a suitable alarm sound warning (for example through light/sound indicators 6 and 7) in case the presence of people and/or animals is detected in the environment before the tolerable ozone concentration level has been reached.

A filtering assembly (not shown) is furthermore preferably provided, apt to be inserted in the path of the airflow and suited to achieve a reduction of the ozone contents. In such case, at the end of the sanitation cycle, the ozone level in the environment can be reduced quickly causing a room airflow to circulate in the filtering assembly.

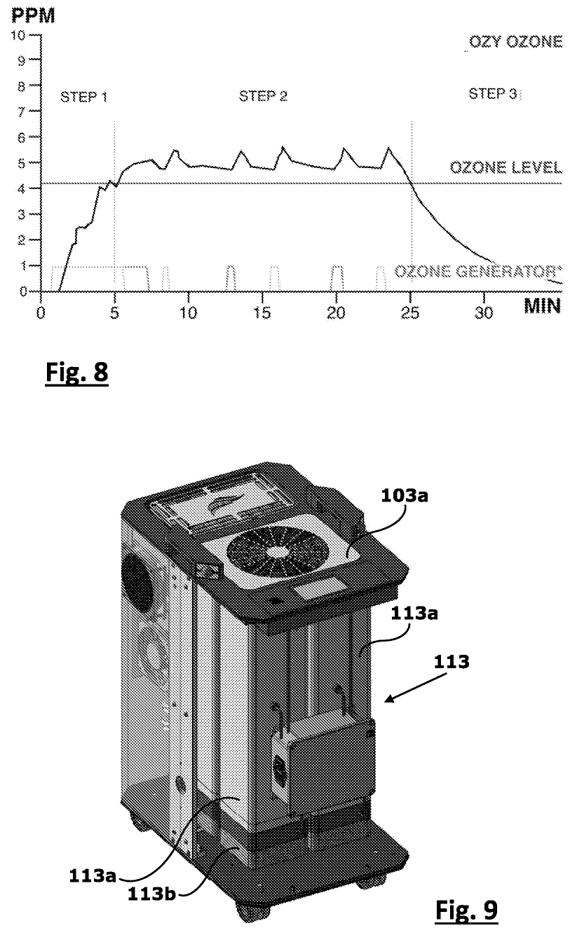

With reference to FIG. 8, control unit C automatically manages the sanitising apparatus with the following exemplifying base cycle:
1. activation of the ozone generators, up until a programmed threshold (horizontal ‘ozone level’ line in FIG. 8) is reached;
2. maintaining the reached ozone level (plateau) for the scheduled time, with the automatic maintaining of the concentration level between two thresholds (min and max), slightly above the scheduled threshold;
3. at the end of the preceding step, possible removal of the excess ozone through activation of the filtering assembly (particle/molecular filter) until reaching the threshold of 0.2 ppm, that is the one useful for the immediate use of the treated environment.

In the diagram of FIG. 8 the trend of the ozone concentration in the environment is clearly shown, in the top part, and correspondingly, in the bottom part, the diagram of the duty cycle of the ozone generator. Please notice that the full operation cycle, compatibly with the volume of the room wherein the sanitising module is caused to operate, it is in the order of 40 minutes. The second maintenance step of the plateau must nevertheless be of at least 20 minutes to obtain the desired effectiveness.

The ozonising module can also be programmed to repeat continuously an operation cycle - for example set for ozone production only at nighttime hours - until it is disabled or the power supply is interrupted.

In addition to the operation cycle for ozone generation and for the obtaining of information of the level thereof, it is possible to have additional information, through the integration of further sensors, which allow to optimise operation.

As an example, the following ones can be installed: temperature sensors, humidity sensors, air quality sensors, oxygen sensors (O₂), carbon monoxide sensors (CO), carbon dioxide sensors (CO₂), particulate sensors (PM2, 5 and PM10), VOC sensors, nitrogen dioxide sensors (NO2), etc.

All the operating and information data can be suitably shown to a user through a graphic interface on a display of a suitably programmed device.

In FIGS. 5-7 an alternative embodiment of the module according to the invention is shown, identified in its entirety by reference number 100.

In this case, too, an O₃ generator assembly 101 is provided, which is the combination of individual components comprising at least an ozone generator 101a and ventilation means with controlled flow rate 101b. In the exemplifying version of FIG. 7 four generators 101a are provided, for example each one with an ozone production of 10 gr/h, each one provided with an own fan with controlled flow rate 101b. The fans can be mounted through a cut-out 107c of carter 107 or they can remain fully within carter 7 and create an airflow with the outside of the module through grids 107b.

The technique used is the one of the so-called “corona plates” but it is possible to use any other system for ozone generation. Generators with different productivity are used which vary depending on the volume of the rooms where the specific apparatus is to be installed.

The layout shown is exemplifying, but one or more “corona plates” can be provided, one or more fans, one or more conveyors of an O₃ flow as well as elements for the wiring and for the connection to a power panel.

Ventilation means 101b are typically in the shape of standard fans (consisting of an electric motor and impeller thereof), of a diameter suitable for the size of the air conveyor, preferably provided with a tachometer generator for the adjustment of the revolution speed. The fan is electrically connected to a control unit C to be able to receive power supply current and adjustment signals. Control unit C is fully similar to the one already described above, preferably also provided with a display V.

Due to the presence of the tachometer generator, it is possible to vary the flow rate and the speed of the air pushed and/or sucked in by the fan according to the signals coming from the control unit. The tachometer generator also serves a diagnostic function, since a feedback signal from the tachometer generator (tachogenerator) to the control unit supplies values usable for detecting the fan operation.

As for the first embodiment, in this case, too, the module furthermore comprises an ozone sensor 102, which is one of the fundamental components.

The addition of a further sensor is provided but not binding with the purpose of making the signalling of the safe level of ozone concentration redundant in the reference volume, supplied independently (electric network or battery) and connected to the control unit of the module, for example in a wireless manner.

Also in this case sensors are provided, as well as presence detectors 108 and function and emergency indicators described for the first embodiment.

A filtering assembly 103 is furthermore provided, which acts as particle/molecular filter suitable for mechanically filtering VOC and particulate up to PM 2.5. The filtering assembly, having active carbon filters or the like, is furthermore used for the quick conversion of O₃ into O₂ through a catalyst at the end or during a break of the sanitising cycle. In such sense, the filtering assembly is also definable as an ozone reducing assembly.

Filtering assembly 103 is provided with dedicated ventilation means 103a, which are operated to cause the room air to flow through filtering assembly 103. The airflow furthermore runs through a grid 107a on carter 107 before or after the entry into filter 103. Ventilation means 103 are controlled by the control unit, so that they are operated only when the work cycle provides it, that is at the end of ozone production.

A sensor automatically determines the filter saturation level, which is communicated for example by means of an application installed on a user’s portable device (smartphone, smartwatch, pda, laptop, ...). The flow rate of the airflow can be provided in the order of nominal 900 m³/h with a fixed speed of the fan, but it is provided to be able to install a speed adjuster to be able to manage smaller flow rates and the resulting optimisation of the noise level.

Preferably filtering assembly 103 is housed in a module compartment which is kept substantially separate from a corresponding compartment where ozone generator assembly 101 is housed, for example through a partition 106a.

A power box 104 is furthermore provided, wherein the lines of the sensors and the power supply lines of the devices converge, so as to make the module independent and operating autonomously. A connection for power supply (for example 220 ACV), equipped with light indicator to indicate voltage presence is a so provided.

In the drawings the control unit 105 of the process is furthermore shown (which can be of a commercial type, Raspberry™ or Arduino™ type or others, or embedded).

As clearly shown, all the components are mounted on a frame 106 provided with ground supports 107 (for example in the shape of swivel caster), enclosed in a carter 108 consisting of elements made of bent metal sheet (for example stainless steel) or of thermoformed plastic materials (for example ABS).

The module can be supplied in different sizes and layouts. For example,
a stand-alone device with a production of 20 - 60 g/h, which acts as portable, wheel-mounted electrical household item, handy to be easily moved from one room to another,
a device with a production of 20 - 60 g/h but conceived to be integrated in suspended ceilings, for example for a version with modular 60 × 60 cm panels; a central control logic (connected to the Internet - Ethernet) can manage all the ozonizers located in the different rooms;
a small-sized device, with a production of about 10 g/h, useful for the transport means meant for miscellaneous use such as ambulances, work means, rental cars; this model can be devoid of particle/molecular filter since the volume to be sanitised is extremely small and the time for the natural conversion of O₃ back into O₂ is considered suitable.

FIG. 9 shows a further embodiment, which is a variant of the one shown in FIGS. 5-7. In this case, instead of filtering assembly 103, a UV modular unit 113 is arranged, consisting of outflow elements 113a wherein UV lamps are installed.

UV modular unit 113 is as described in Italian application no. 102021000007910 which is here recomprised by reference.

Unit 113 consists of a plurality of flow elements located side by side, each one making up a UV sanitising module 113a, consisting of elongated hollow bodies, within which UV lamps (not shown) are housed. Within each sanitation module 113a an airflow is caused to flow, which is pushed by venting means 103a and is preferably filtered by filtering elements 113b arranged at the opposite end of venting means 103a.

Unit 113 can be used for integrating the air sanitising effect obtained with the ozonising part, or it can be used as ozone assembly, that is for converting the molecules of O₃ into O₂ molecules thanks to the action of UV radiations produced by the UV lamps (with a similar function to that of filtering assembly 103).

According to a variant (not shown), the module according to the invention is part of a more extended control system wherein two or more ozonising modules are installed, located in various rooms.

The control system provides an online and off-site (such as Amazon AWS) central control server connected to a wider geographic network (WAN) for example Internet, so as to be able to receive the remote controls and transmit them to the individual modules. Modules 1 can be of the electrical household item type or there can be a plurality of mutually interconnected modules 1, on the same LAN WiFi network in turn connected to a WAN network. Modules 1 are located in various rooms of a company, of other structured, private and public buildings, but also of means of transport such as trains, ships and airplanes for example.

Such a system allows to manage and coordinate the various modules, both with punctual controls and in an automated manner through suitable scheduling. At the same time the system allows to collect a plurality of data from sensors onboard the modules, to have an overall picture of the operation and of the sanitation condition of the various rooms.

On the server it is preferably provided also a database wherein the operation data of the individual modules are stored, the corresponding time scheduling and the data of the users associated with and enabled to access the various modules.

The database can be structured so that each user be associated with one or more buildings (home, office, gym, ...), and each building can have one or more rooms. Each room then corresponds to one or more modules.

In this way, in the database each user is enabled to programme one or more modules, each of them identified in the system by its own univocal ID (for example corresponding to the MAC address of the network communicator module).

The server is designed to manage the data of the modules, viewable through an application for web browser or an application for portable devices (for example for Android™ or iOS™ operating systems), or the control signals apt to be sent to the various modules through the same applications. In this way a centralised management of all the possible modules is obtained, with no number nor location limit.

Through the system server the suitably enabled user can obtain (through notifications on the application or the receipt of a text message or an e-mail) real-time information about the sanitation level of a room where he or she is going to or of the need to vacate a certain room to allow the scheduled operation of the ozonising module.

The information available on the server can be used also for other complementary functions. For example, it is possible to integrate in the system controlled commands of room access locks, so as to prevent access to the rooms wherein sanitising by the corresponding module is under way. Furthermore, it is possible to integrate in the system controls which control the opening/closing of windows of the room and/or the turning on/off of fans or air conditioning systems and/or other systems, so as to automatically air the rooms at the end of the sanitising process.

As can be guessed from the above-reported description, the module and the system according to the invention perfectly meet the objects set forth in the preamble. As a matter of fact, by integrating in the same module an ozone generator, suitably programmed and controlled by means of ozone concentration sensors and presence sensors, a solution to the permanent sanitising of rooms is supplied, without risks for the occupiers and with the opportunity of suitably adjusting the desired operation.

By the system of the invention it is furthermore possible to suitably coordinate a series of ozonising modules in structured buildings and thus supply the users with a safe and healthy experience in work and domestic rooms.

It is understood that the invention must nevertheless not be considered limited to the particular embodiments described and illustrated, but that different variants are possible, all within the reach of a person skilled in the field, without departing from the scope of protection of the invention, which is only defined by the following claims.

For example, the ozonising modules can be provided with a modular structure, where the various components are easily engageable (in a plug&play manner) with a main motherboard also by non-expert users. In this way an essential module can be supplied, provided with the sole ozone generator and sensor, which can be scaled and integrated by the user, who progressively purchases also the other accessory components and then can insert a plurality of modules into a system.

Furthermore, the various components can be replaced by similar elements equivalent to the ones described. For example, instead of a WiFi module it is possible to provide an Ethernet card for connection to a cable network or equivalent connection unit.

1. Ozonising module (1, 100) comprising a housing (1a, 1b, 107) wherein an ozone generator assembly (3, 101) is housed, characterised in that it furthermore comprises
at least an ozone sensor (4, 102) connected to a control unit (C) apt to operate said ozone generator (3, 101) based on time scheduling and on a comparison between an ozone threshold concentration and a concentration signal coming from said ozone sensor (4, 102),
ozone reducing means consisting of a filtering assembly with catalytic action (103) or of a reducing assembly employing UV sources (113) through which an airflow of room air is caused to flow by means of respective controlled ventilation means (103a).

2. The ozonising module as in claim 1, wherein said ozone sensor (4) is connected to said control unit by communication means (W).

3. The ozonising module as in claim 2, wherein said communication means (W) comprise a wireless data communicator and said ozone sensor (4, 102) is self-contained and apt to be moved away from said ozone generator (3, 101).

4. The ozonising module as in claim 1, wherein a presence detector (8, 108) is furthermore provided.

5. The ozonising module as in claim 1, wherein said ozone generator (3, 101) is housed in a conveyor duct (32) provided with a fan (33, 101b) at an inlet opening thereof and with a deviator duct (34, 107b) at an exit threof, apt to create an airflow of ozone-rich air directed outwards of said housing (1a, 1b, 107).

6. The ozonising module as in claim 1, wherein light/acoustic/visual alarms (6, 7, V) of the operation of the ozone generator (3, 101) are furthermore provided, arranged visibly outside of said housing (1a, 1b, 107).

7. The ozonising module as in claim 1, wherein one or more of the following sensors are furthermore provided: smoke sensor, temperature sensor, humidity sensor, air quality sensor, oxygen sensor (O₂), carbon monoxide sensor (CO), carbon dioxide sensor (CO₂).

8. The ozonising module as in any one of the preceding claims, wherein said control unit (C) is provided with a programmable unit.

9. Management system of a plurality of modules (1, 100) as in claim 1, wherein each module has communication means (W) connected to a local network (LAN), connected to a geographical network (WAN), through which control units of said modules (1, 100) are managed by a common server.

10. Management system as in claim 9, wherein said common server comprises a database wherein operation data of each module as well as identification data of users associated with and enabled to programme said modules (1, 100) are stored.
